(19) United States
(12) Patent Application Publication
(10) Pub. No.: US 2009/0297510 A1
(43) Pub. Date: Dec. 3, 2009

(54) INHIBITION OF CALPAIN REDUCES ALLERGIC INFLAMMATION

(76) Inventor: Tong-Jun Lin, Halifax (CA)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 12/323,381

(22) Filed: Nov. 25, 2008

(30) Foreign Application Priority Data
May 9, 2008 (CA) 2631071

(51) Int. Cl. A61K 39/395; A61K 38/02; A61K 31/7088; A61P 37/00; A61P 37/08; A61P 29/00

(52) U.S. Cl. 424/133.1; 514/2; 514/44 R; 514/44 A

(57) ABSTRACT

The present invention provides for the use of a calpain inhibitor for the treatment of allergy diseases, inflammatory diseases and autoimmune diseases. In particular, it has been found that inhibition of calpain within mast cells decreases the release of chemical mediators into the surrounding environment, resulting in a decreased local inflammatory response. The present invention also provides for a method of decreasing IgE-dependent mast cell degranulation, mast cell mediator release and cytokine production in mast cells, comprising the step of administering to the mast cells an effective amount of a calpain inhibitor.

### FIELD OF THE INVENTION

The present invention relates to the treatment of allergy and/or inflammatory diseases through inhibition of the enzymatic activity of the cellular protease, calpain, in mast cells.

### BACKGROUND OF THE INVENTION

Allergic diseases such as asthma, rhinitis or atopic dermatitis affect at least 8%-16% of the population with the annual economic burden of 12.7, 1.2, and 3.8 billion dollars, respectively, in the United States and are also a major health burden in Canada and world-wide. Mast cells play a central role in allergic inflammation. This is because antigen (Ag)-induced aggregation of IgE-FcεRI on mast cells initiates a cascade of signaling events leading to mast cell mediator secretion. The goal of this study is to investigate the signaling mechanisms controlling FcεRI-dependent mast cell mediator secretion with a focus on calpain.

Mast cells play diverse and significant roles. For example, mast cells are involved in mediating first line immune responses of the innate immune system seen in response to allergens and/or bacterial infections. Mast cells also contribute to the recruitment of other inflammatory cells, such as neutrophils and T cells, which initiates a second line immune response.

Accordingly, mast cells play an essential role in allergic reactions and inflammation. Many of the mast cell's effects are mediated through secretion of mast cell mediators upon activation via Antigen (Ag)-induced FcεRI aggregation. Mast cell mediators can be broadly divided into preformed mediators, cytokines and lipid mediators. Preformed mediators are presynthesized and stored in mast cell granules and are secreted by degranulation. Examples of preformed mediators include histamine and serotonin; proteolytic enzymes, such as tryptase that can destroy tissue or cleave complement components or coagulation components; heparin or chondroitin sulfate, which are anticoagulants; chemotactic factors, such as eosinophil chemotactic factor of anaphylaxis and neutrophil chemotactic factor. During mast cell activation, these mediators are released into the environment leading to acute and immediate immune responses.

Upon Antigen activation, mast cells also produce and secrete another important category of mediators through de novo synthesis, namely, newly synthesized mediators. These include cytokines, such as TNF and IL-6, as well as lipid mediators, such as prostaglandin D₂, platelet-activating factor and leukotrienes C₄ and D₄.

Mast cell activity is necessary and desirable in healthy individuals. Unwanted mast cell activity may be a component of a wide variety of disease states and/or their symptoms. In such instances, it is often desirable, from a therapeutic or preventative standpoint, to reduce or eliminate mast cell activity. For example, numerous immune mediated diseases involve the release by mast cells of cytokines, chemokines and other factors. These mediators often recruit additional immune cells such as lymphocytes to the site of inflammation. The end result of this is potentially additional numerous immune-related diseases.

Traditional treatment for mast cell-based inflammation typically encompasses three possible options. Avoidance or reduced exposure to the allergen, which can be very difficult when children are involved. Allergen immunotherapy, which has been found to only work for some allergens. And finally, pharmacotherapy, which appears to be the most effective line of defense. Medication should be based around either preventing the release of mediators from mast cells, or, stop and/or alleviate the effect that the mediators have on the surrounding tissues. Clearly, the most common medication in this regard are anti-histamine medications, exemplified by diphenhydramine, and the more recent loratidine.

The main downfall to anti-histamine medication is that they only counteract the direct effect of histamine in the surrounding tissue. Although histamine is associated with many of the effect of allergy and inflammation, there are many other mediators that are released by mast cells which also produce undesirable results. Accordingly, the effects of these other mediators, as indicated above, remain unaffected by anti-histamines, and contribute significantly to the pathophysiology of many allergic and inflammatory diseases.

The initiation of mast cell degranulation can be traced to FcεRI aggregation. FcεRI on mast cells is comprised of α, β, and 2 γ chains. The α chain is primarily responsible for IgE binding, while the β and γ chains are critical for signal transduction. Antigen (Ag)-binding to IgE mediates aggregation of FcεRI which in turn initiates a cascade of molecular events leading to the release of both preformed and newly synthesized mediators. Two major mechanisms of protein modification, reversible protein phosphorylation controlled by protein kinases and phosphatases, and irreversible protein cleavage induced by proteases, play a fundamental role in such signal transduction.

Two major protein kinase families, protein tyrosine kinases (PTK) and protein Ser/Thr kinases, play an essential role in FcεRI-mediated mast cell responses. The early molecular events immediately following FcεRI aggregation are mediated by PTK, such as Lyn, Fyn and Syk, and followed by activation of many Ser/Thr kinases such as MAP kinases (p38, JNK, and ERK1/2) and IKK-IκB. These lead to activation of transcription factors including NF-κB. An essential role of NF-κB in allergic inflammation has been demonstrated because NF-κB p50-deficient mice have an inhibited inflammatory response (Yang et al. J Exp Med 188, 1739-50 (1998); Das et al. Nat Immunol 2, 45-50 (2001)) Thus, regulation of protein kinases may serve as a potential approach for the management of IgE-dependent mast cell activation.

Irreversible protein cleavage by proteases is an essential tool utilized by cells to transduce signals. One of the best-characterized proteases is cysteine protease family including caspases and calpains, etc. A role for caspases in apoptosis has been well recognized. Mast cells also utilize proteolysis mechanism to transduce signals from membrane to nucleus. However, little is known about the nature of the proteases involved in FcεRI-induced signaling. Calpains are unique cysteine proteases that require Ca2+ for activity. Calpains cleave substrate proteins localizing near membranes and cytoskeletons in a limited manner. The calpain family is composed of calpain 1 to calpain 14. (Sato et al. Biol. Chem. 382, 743-51 (2001); Huang Y. and Wang K. K. Trends Mol. Med. 7, 355-62 (2001)) Major calpain species are calpain 1 (μ-calpain) and calpain 2 (m-calpain) which require micro and millimolar concentrations of Ca2+ for activation, respectively. Both calpain 1 and 2 require calpain 4 for activities. It is well known that FcεRI aggregation in mast cells induces a rapid increase of intracellular Ca2+ levels, which is essential for mast cell activation. (Hoth M. et al. Ann. NY Acad. Sci. 707, 198-209 (1993)) Furthermore, a number of well-characterized calpain substrates are important signaling molecules involved in FcεRI-induced mast cell activation, such as PKC (Sato K. and Kawashima S. Biol. Chem. 382, 743-51 (2001)), Iκb (Pianetti S. Oncogene 20, 1287-99 (2001)), PP2B (Sato K. and Kawashima S. Biol. Chem. 382, 743-51 (2001)), and SNAP23 (Rutledge T. W. and Whiteheart S. W. J. Biol. Chem. 277, 37009-15 (2002); Guo Z. et al. Cell 94, 537-48 (1998)). In addition, it has been shown that active calpain alters the balance between protein kinase and phosphatase activities. (Robles E. et al. Neuron 38, 597-609 (2003)) Therefore, calpain may well play a role in FcεRI-induced mast cell activation and it is logical to examine the contribution of calpain in IgE-dependent mast cell activation. We have found that inhibition of calpain reduced IgE-dependent mast cell degranulation mediator release and cytokine production, processes that are vital to the inflammatory response.

The present invention is based on the discovery that inhibitors of calpain are able to decrease allergen-induced mast cell activation leading to a subsequent decrease in the release of inflammatory mediators. These molecules can be delivered alone or in combination with other agents that possess anti-inflammatory properties.

Therefore, it is an object of the present invention to provide a safer and more effective treatment for allergy and/or inflammatory diseases.

### SUMMARY OF THE INVENTION

Unless defined otherwise, all of the scientific and technical terms used herein maintain the standard meaning as to be understood by one of ordinary skill in the art.

According to an aspect of the present invention there is provided a method of decreasing IgE-dependent degranulation of mast cells, comprising the step of decreasing the activity of calpain within mast cells.

According to another aspect of the present invention, there is provided a method of decreasing cytokine production by mast cells, comprising the step of decreasing the activity of calpain within mast cells.

According to yet another aspect of the present invention, there is provided a method of decreasing mediator release from mast cells, comprising the step of decreasing the activity of calpain within mast cells.

According to a further aspect of the present invention, there is provided a method of therapeutically treating an allergic disease, inflammatory disease or autoimmune disease in a subject in need thereof, comprising the step of administering to said subject a pharmaceutical composition comprising an effective amount of at least one inhibitor of calpain and a pharmaceutically acceptable carrier.

Preferably the activity of calpain is decreased by utilizing a calpain inhibitor.

According to another aspect of the present invention, there is provided the use of a pharmacologically acceptable calpain inhibitor for the treatment of allergic diseases, inflammatory diseases or autoimmune diseases.

The compounds used in the present invention are those that inhibit or reduce the activity of calpain. In this context, the term “inhibitor” refers to any molecule that, when administered to the mast cells in an effective amount, is able to cause inhibition and/or reduction of the enzyme activity. Inhibition and/or reduction of activity refers to a lower level of measurable activity relative to a control experiment in which the enzyme, cell or subject is not treated with the test compound. In particular embodiments, the inhibition or reduction in the measured activity is at least a 1% reduction and/or inhibition. A person of skill in the art will appreciated that reduction or inhibition of the measure activity of at least 20%, 50%, 75%, 90% or 100% or any integer between 1% and 100%, may be preferred for particular applications. An inhibitor may take the form of a pharmaceutical. However, decreasing and/or reducing the activity of calpain can also be obtained by negatively augmenting the expression profile of calpain within a cell, through, for example, using antisense oligonucleotides or RNAi. Accordingly, these methods are also considered to be encompassed by the definition of “inhibitor”.

By “pharmacologically acceptable”, what is meant is a material which is not biologically or otherwise undesirable, i.e., the material may be administered to a subject without causing any undesirable biological effects or interacting in a deleterious manner with any of the components of the composition in which it is contained.

According to the present invention, an inhibitor of calpain can be used for the manufacture of a drug preparation for the treatment of disease relating to mast cell mediator secretion, comprising an effective amount of said inhibitor of calpain.

As used herein, the terms “treat” or “treatment” are used interchangeably and are meant to indicate a postponement of development of symptoms and/or a reduction in the severity of such symptoms that will or are expected to develop, or that have already developed.

The terms “effective amount” or “pharmaceutically effective amount” refer to a nontoxic but sufficient amount of the agent to provide the desired biological result. This result can be an amount that is able to effectively decrease the activity of the calpain enzyme. That result can also be reduction and/or alleviation of the signs, symptoms, or causes of a disease, or any other desired alteration of a biological system.

The method of delivery of the drug preparation to a subject in need thereof is not particularly limiting, and may be administered orally, parenterally, intravascularly, intranasally, intrabronchially, sublingually, transdermally, or rectally. Alternatively, additional modes of drug delivery not explicitly noted herein are also contemplated.

Several pharmacological inhibitors of calpain are known which are contemplated within the scope of the present invention. These inhibitors include, but are not limited to, peptidyl epoxides, such as E64, E64C and E64D; peptidyl aldehydes, such as leupeptin, calpeptin, SJA-6017 or calpain inhibitor VI, MDL-28170 of calpain inhibitor III, ALLN or calpain inhibitor I and ALLM or calpain inhibitor II; peptidyl α-ketoamides, such as AK275, AK295, CX295, BSF 409425, ZLLYCH2F or calpain inhibitor IV, N-(1-carbamoyl-1-oxohex-1-yl)-2-[E-2-(4-dimethyl-aminomethylphenyl)-ethen-1-yl]benzamide (5b), calpain inhibitor X or Z-L-Abu-CONH-ethyl, calpain inhibitor XII or Z-L-Nva-CONH—CH₂-2-Py, A-705253 or CAL 9961, naphtalene 19c and A-705239; non-peptide inhibitors, such as α-mercaptoaclylic acids, PD150606, PD151746, AT A (aurintricarboxylic acid) and carboxamide derivatives e.g. quinoline carboxamides. These inhibitors can be used alone or in any combination, while still falling within the scope of the present invention.

According to the present invention, it is also contemplated that calpain activity can be diminished through knockdown or decrease of its expression level. Knockdown or decrease of the expression level of calpain can be obtained through a variety of known methods.

In another embodiment of the present invention, there is the use of a pharmacologically acceptable effective amount of an antisense oligonucleotide having a sequence capable of binding specifically with any sequence of genomic DNA or an mRNA molecule which encodes calpain, so as to prevent transcription or translation of calpain mRNA. By “antisense”, what is meant is a composition containing a nucleic acid sequence which is complementary to the “sense” strand of a specific nucleic acid sequence. Once introduced into a cell, the complementary nucleotides combine with endogenous sequences produced by the cell to form duplexes and to block either transcription or translation. (Alama et al. Pharmacol. Res 36:171-178 (1997); Crooke, S. T. Adv. Pharmacol. 40:1-49 (1997)) Antisense sequences can be any nucleic acid material, including DNA, RNA, or any nucleic acid mimics or analogs. (Lee et al. Biochemistry 37:900-1010 (1998)) Delivery of antisense sequences can be accomplished in a variety of ways, such as through intracellular delivery using a recombinant vector.

Antisense oligonucleotides of about 15 to 25 nucleic acid bases are typically preferred as such are easily synthesized and are capable of producing the desired inhibitory effect. Molecular analogs of antisense oligonucleotides may also be used for this purpose and can have added advantages such as stability, distribution, or limited toxicity, all of which are advantageous in a pharmaceutical product. In addition, chemically reactive groups, such as iron-linked ethylenediamine-tetraaetic acid (EDTA-Fe), can be attached to antisense oligonucleotides, causing cleavage of the RNA at the site of hybridization. These and other uses of antisense methods to inhibit the in vitro translation of genes are well known in the art. (Marcus-Sekura Anal. Biochem. 172:289 (1988))

In a further embodiment of the present invention, there is the use of RNAi methodologies, so as to prevent transcription or translation of calpain mRNA. By “RNA interference (RNAi)”, what is meant is the administration of a nucleic acid molecule (e.g., antisense shRNA, siRNA, dsRNA), regardless of length, that inhibits the expression of a calpain gene. Typically, the administered nucleic acid molecule contains one strand that is complementary to the coding strand of an mRNA of a calpain gene. RNAi is a form of post-transcriptional gene silencing initiated by the introduction of double-stranded RNA (dsRNA) or antisense RNA. Preferably, RNAi is capable of decreasing the expression of calpain in a cell by at least 10%, 20%, 30%, or 40%, more preferably by at least 50%, 60%, or 70%, and most preferably by at least 75%, 80%, 90%, 95% or more. The double stranded RNA or antisense RNA is at least 10, 20 or 30 nucleotides or more, in length. Other preferred lengths include 40, 60, 85, 120, or any length of consecutive nucleotides that are complementary to a calpain mRNA or DNA, and may be as long as a full length calpain gene or mRNA. The double stranded nucleic acid may contain a modified backbone, for example, phosphorothioate, phosphorodithioate, or other modified backbones known in the art, or may contain non-natural internucleoside linkages. In one preferred embodiment, short 21, 22, 23, 24, or 25 nucleotide double stranded RNAs are used to down regulate calpain expression. Such RNAs are effective at down-regulating gene expression in mammalian tissue culture cell lines (Elbashir et al. Nature 411:494-498 (2001)). The further therapeutic effectiveness of this approach in mammals was demonstrated in vivo by McCaffrey et al. (Nat 418:38-39 (2002). The nucleic acid sequence of a calpain gene can be used to design small interfering RNAs that will inactivate a calpain gene and that may be used, for example, as a therapeutic to treat a wide variety of inflammatory and mast cell-related diseases.

By “small interfering RNAs (siRNAs)”, what is meant is an isolated dsRNA molecule, preferably greater than 10 nucleotides in length, more preferably greater than 15 nucleotides in length, and most preferably 18 to 25 nucleotides in length that is used to identify the target gene or mRNA to be degraded. A range of 19 to 25 nucleotides is the most preferred size for siRNAs. siRNAs can also include short hairpin RNAs in which both strands of an sirRNA duplex are included within a single RNA molecule. siRNA includes any form of dsRNA (proteolytically cleaved products of larger dsRNA, partially purified RNA, essentially pre RNA, synthetic RNA, recombinantly produced RNA) as well as altered RNA that differs from naturally occurring RNA by the addition, deletion, substitution, and/or alteration of one or more nucleotides. Such alteration can include the addition of non-nucleotide material, such as to the end(s) of the 21 to 23 nucleotide RNA or internally (at one or more nucleotides of the RNA). In a preferred embodiment, the RNA molecule contains a 3′hydroxyl group. Nucleotides in the RNA molecules of the present invention can also comprise non-standard nucleotides, including non-naturally occurring nucleotides or deoxyribonucleotides. Collectively, all such altered RNAs are referred to as analogs of RNA. siRNAs of the present invention need only be sufficiently similar to natural RNA in that it has the ability to distinguish or identify with RNAs are to be degraded.

The siRNA may be encoded by a nucleic acid sequence, and nucleic acid sequence can also include a promoter. The nucleic acid sequence can also include a polyadenylation signal. In some embodiments, the polyadenylation signal is a synthetic minimal polyadenylation signal. The RNA duplex of the siRNA may be constructed in vitro using synthetic oligonucleotides.

The exact portion of a calpain gene, or which calpain gene itself, which is targeted through either the antisense oligonucleotide or RNAi strategy is well within the state of the art. A person of ordinary skill would be able to locate the sequence of the desired calpain gene or mRNA through one of many nucleotide sequence databases, such as GenBank. The only requirement is that the calpain gene selected represents a calpain isoform that is expressed in mast cells, e.g. calpain 1, calpain 2 or calpain 4, and that the section of the gene that is targeted will result in an overall decrease in cellular calpain enzymatic activity of at least about 1%. Preferably, the decrease in calpain enzymatic activity is at least about 10%. More preferably the decrease in calpain enzymatic activity is at least about 25%, and most preferably, the decrease in calpain enzymatic activity is at least about 50%.

In accordance with the present invention, it is envisioned that inhibition of calpain, such as by administration of a calpain inhibitor, is useful for the treatment of diseases associated with mast cell activation. This extends to a wide variety of potential maladies, including allergy diseases, which are exemplified by allergic rhinitis; atopic eczema; asthma; conjunctivitis; and anaphylaxis, brought on, for example, by food and/or drug allergies.

It is also contemplated that inhibition of calpain activity in mast cells, such as by administration of a calpain inhibitor, will benefit autoimmune diseases. Non-limiting examples of autoimmune diseases are multiple sclerosis, psoriasis, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, vasculitis, Sjogren's syndrome, nodular panarteritis, autoimmune enteropathy, as well as proliferative glomerulonephritis.

In another embodiment of the invention, there is provided a pharmaceutical composition which is capable of decreasing degranulation of mast cells. Said composition comprises a molecule capable of decreasing the enzymatic activity of calpain and at least one additional molecule that possesses therapeutic properties directed to alleviate allergy and/or treat inflammation when administered to a subject in need.

According to the present invention, it is contemplated that an inhibitor of calpain may be co-administered to a subject in need with at least one additional molecule which will either enhance the activity of the inhibitor or compliment its activity or use in treatment. Such additional molecules may produce a synergistic effect with the inhibitor, or may minimize potential side effects.

The additional molecule to be co-administered may be an histamine receptor antagonist selected from the group consisting of tricyclic dibenzoxepins, such as doxepin hydrochloride; ethanolamines, such as carbinoxamine maleate, clemastine fumarate, diphenhydramine HCl and dimenhydrinate; ethylenediamines, such as pyrilamine maleate, tripelennamine HCl and tripelennamine citrate; alylamines, such as acrivastine, chlorpheniramine maleate and brompheniramine maleate; piperazines, such as cetirizine hydrochloride, hydroxyzine HCl, hydroxyzine pamoate, cyclizine HCl, cyclizine lactate and meclizine HCl; phenothiazines, such as promethazine HCl; piperidines, such as levocabastine hydrochloride, loratadine, desloratidine, ebastine, mizolastine, fexofenadine, cyproheptadine and phenindamine tartrate; and phthalazinones, such as azelastine hydrochloride.

Alternatively, or in addition to the histamine receptor antagonist, the additional molecule to be co-administered may be an immunomodulatory agent selected from the group consisting of glucocorticoids, such as cortisone, dexamethosone, hydrocotrisone, methylprednisolone, prednisolone, prednisone, and budesonide; immunosuppressants, such as azathioprine, cyclophosphamide, FTY720, tacrolimus, cyclosporine, and methotrexate; aminosalicylates; steroid hormones; non-steroidal anti-inflammatory drugs (NSAIDS), such as ibuprofen, naproxen, Cox-2 inhibitors and salicylate; sympathomimetics; and analgesics, such as acetaminophen, oxycodone, tramadol. Other therapeutic compounds that are envisioned within the scope of the present invention for co-administration are omalizumab; leukotriene receptor antagonists, such as montelukast and zafirlukast; leukotriene synthesis inhibitors, such as zileuton; cromolyn; and nedocromil.

While a number of calpain inhibitors and means to inhibit calpain activity are described herein and known, it is understood that many others may be used, while still existing within the scope of the present invention. The invention described herein is intended to include the use of currently known calpain inhibitors as well as those discovered subsequently. The assays described below and those to a person of skill in the art enable other calpain inhibitors that are useful for preventing or treating inflammatory and/or allergy conditions to be readily identified and developed.

### BRIEF DESCRIPTION OF THE DRAWINGS

The present invention will be described in detail below, in which reference is made to the following Drawings.

FIG. 1 is a series of graphs illustrating that inhibition of calpain reduced IgE-dependent mast cell degranulation. In particular, mouse bone marrow-derived mast cell were sensitized with anti-trinitrophenol (TNP) IgE. Cells were treated with various calpain inhibitors or transfected with siRNA directed to calpain 4 and subsequently stimulated with antigen TNP-bovine serum albumin (BSA) (10 ng/ml). Mast cell degranulation was determined by the release of β-hexosaminidase.

FIG. 2 illustrates that inhibition of calpain reduced IgE-dependent passive cutaneous anaphylaxis. In particular, mice were injected locally in the ear tissue with anti-dinitrophenol (DNP) IgE (20 ng/mouse) a day before. Mice in the treatment group received 0.965 mg/mouse of calpain inhibitor III via intraperitoneal injection. Control group received diluent dimethyl sulfoxide (DMSO) only. One hour later, animals were injected with 100 μg of DNP-BSA in 200 μl 0.5% Evan blue dye via tail vein injection. Thirty minutes later, ear tissues were collected. Evan blue dyes were extracted and measure at 620 nm.

FIG. 3 is a series of graphs which illustrate that inhibition of calpain reduced IgE-dependent mast cell cytokine production. In particular, mouse bone marrow-derived mast cells were sensitized with anti-trinitrophenol (TNP) IgE. Cells were treated with calpain inhibitors and subsequently stimulated with antigen TNP-BSA (10 ng/ml). Mast cell cytokine production was determined by ELISA.

FIG. 4 is a western blot illustrating that inhibition of calpain reduced IgE-dependent JNK phosphorylation in mast cells. In particular, mouse bone marrow-derived mast cells (60 million) were sensitized overnight with 90 ml fresh culture medium and 30 ml TIB141-conditioned medium enriched in IgE directed against trinitrophenyl (TNP). Cells were then washed twice with RPMI 1640 and resuspended in 10% FBS-RPMI 1640 at 1 million cells/ml. Five ml of cells were aliquoted for each treatment. Calpain inhibitor 3 (CI3) or DMSO was added and to the sample and incubated at 37° C. in a 5% CO₂ incubator for 1 hour. Then, TNP-BSA (10 ng/ml) was added to stimulate mast cells for various times as indicated. After incubation, cells were transferred to a 50 ml tube, and centrifuged at 500×g for 5 min at 4° C. Supernatants were discarded and cell pellets were kept on ice. Cell pellets were lysed with 30 μl ice-cold RIPA lysis buffer for each condition. Lysates were transferred to 1.5 ml tubes, vortexed for 10 seconds at the highest setting, pipetted 20 times, vortexed for 10 seconds again, and incubated for 20 minutes on ice. Subsequently, lysates were centrifuged at 15,000×g for 10 minutes in a cold room. Supernatants were then transferred to new tubes and used for Western blotting with antibodies to phospho-IκB, total IκB, phospho-p38, total p38, phospho-JNK, total JNK, phospho-ERK, total ERK and actin.

### DETAILED DESCRIPTION OF THE INVENTION

The invention will be illustrated below by way of the following Examples. These examples are not to be taken as limiting the scope of the present invention, but should be interpreted as exemplary modes of carrying out the present invention.

Mast cell mediators released by degranulation play a key role in induction of acute allergic response. Mast cell degranulation can be determined by the release of β-hexosaminidase in vitro. In vivo, an animal model of FcεRI-mediated passive cutaneous anaphylaxis (PCA), which is characterized by an increase of vascular permeability, has been well accepted for the examination of the role of mast cells in acute allergic responses. In this study we used both in vitro and in vivo model to examine an effect of calpain inhibition on mast cell degranulation.

### EXAMPLES

### Mast Cell Culture

Mast cell cultures were maintained at 37° C. in a sterilized, humidified atmosphere containing 5% CO₂. Murine primary cultured bone marrow-derived mast cells (BMMC) were harvested from the femurs and tibias of C57BL/6 mice from Charles River Laboratories (Montreal, PQ) and maintained in RPMI 1640 medium supplemented with 20% WEHI-3B conditioned medium, 10% FBS, 50 units/ml each of penicillin and streptomycin and 50 μM β-mercaptoethanol. Following 4-5 weeks of culture, mast cell purity of >98% was achieved as assessed by toluidine blue staining (pH=1.0) of fixed cytocentrifuged preparations. Mature mast cells were identified by their morphological features and granule prevalence.

### Murine Mast Cell Sensitization

One day (20-24 hr) prior to experimental immunological activation, BMMC were passively sensitized. Briefly, cells were resuspended in fresh complete medium supplemented with TIB141-conditioned medium enriched in IgE directed against trinitrophenyl (TNP) at a ratio of 3:1. BMMC were typically sensitized at 0.5 million/ml. Following sensitization, experimental groups were washed extensively with RPMI 1640 supplemented with 10% FBS alone, resuspended at higher density (1-5 million/ml) in wash medium and activated by the addition of 10 ng/ml TNP-BSA.

### Example 1

### Mast Cell Degranulation Assay

### β-Hexosaminidase Assay

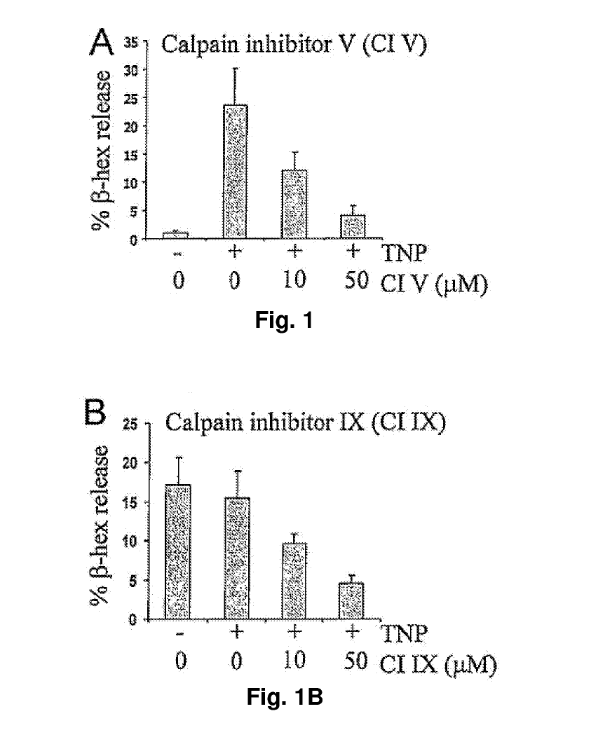

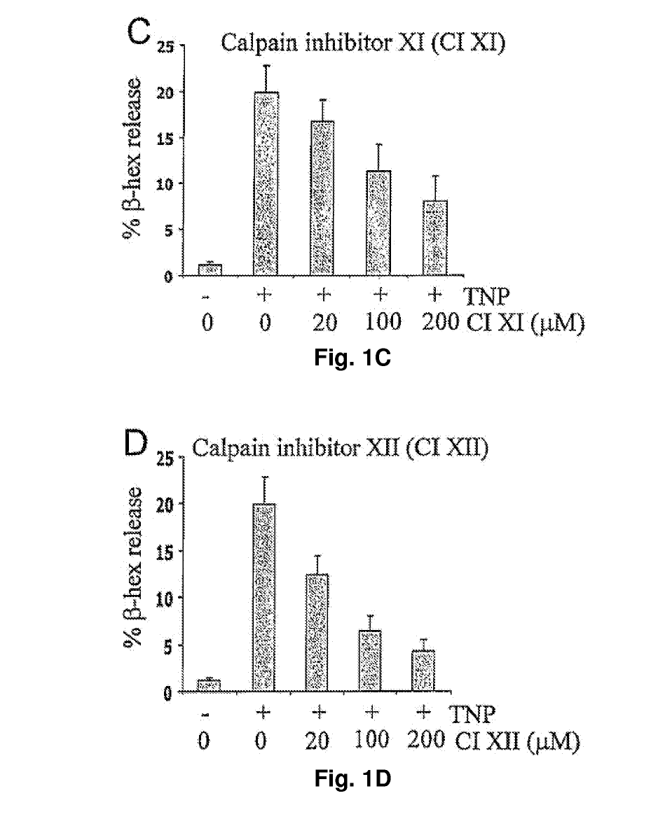

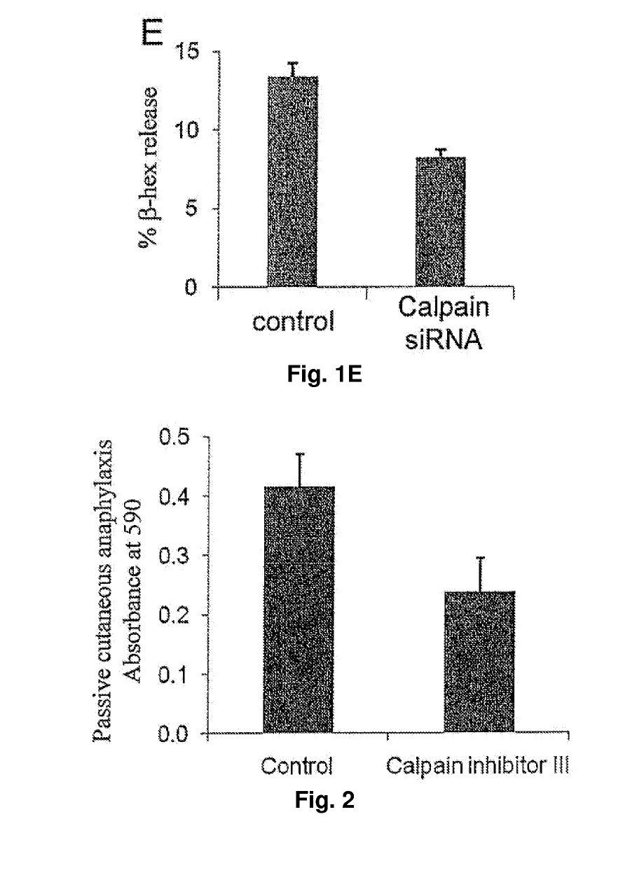

After sensitization with IgE, Mouse bone marrow-derived mast cells (BMMC) were resuspended in Hepes tyrode buffer at a density of 0.5 million cells/ml and a total of 200 μl per test was aliquoted to Eppendorf tubes. Cells were incubated with calpain inhibitors (FIG. 1A, calpain inhibitor V; FIG. 1B, calpain inhibitor IX; FIG. 1C, calpain inhibitor XI; FIG. 1D, calpain inhibitor XII; FIG. 1E, calpain knockdown with siRNA) for 1 h at 37° C. and subsequently, further stimulated with TNP-BSA (10 ng/ml) for 20 min. β-Hexosaminidase, as a measure of degranulation, was measured in both supernatant and pellet fractions using a previously reported method. (Schwartz et al. J. Immunol. 123, 1445-50 (1979)) Briefly, 50 μl of each sample was incubated with 50 μl of 1 mM ρ-nitrophenyl-N-acetyl-β-D-glucosaminide (Sigma) dissolved in 0.1 M citrate buffer, pH 5, in a 96-well microtiter plate at 37° C. for 1 h. The reaction was stopped with 200 μl/well of 0.1 M carbonate buffer, pH 10.5. The plate was read at 405 μm in an ELISA reader.

As can be seen from FIG. 1, these results indicate that calpain specific inhibitors are effective at reducing mast cell degranulation, providing a means for reducing these effects during allergy and/or inflammation disorders.

### Example 2

### Passive Cutaneous Anaphylaxis

To examine the role of calpain in FcεRI-mediated passive cutaneous anaphylaxis in mice, dorsal sides of the ears of Balb/c mice were injected intradermally with 20 ng anti-DNP IgE (both left ears) in a 20 μl volume using a 30-gauge needle. After 24 h mice in the treatment group received 0.965 mg/mouse of calpain inhibitor III via intraperitoneal injection. Control group received diluent dimethyl sulfoxide (DMSO) only. One hour later mice in both treatment and control groups were challenged with 100 μg Ag (DNPBSA) in 200 μl 0.5% Evans blue dye i.v. Mice were sacrificed 30 min after the Ag challenge. For quantitation of Evans blue dye extravasation as a measure of anaphylaxis associated vascular hyperpermeability, 8-mm skin specimens were removed from the ears of mice, minced in 2 ml formamide, and incubated at 80° C. for 2 h in water bath to extract the dye. The absorbance of the extracted dye was read at 620 nm.

These results indicate that specific inhibition of calpain is effective at reducing allergen-induced mast cell degranulation and local inflammation, providing a means for reducing these effects during allergy and/or inflammation disorders.

### Example 3

### Cytokine Release Experiments

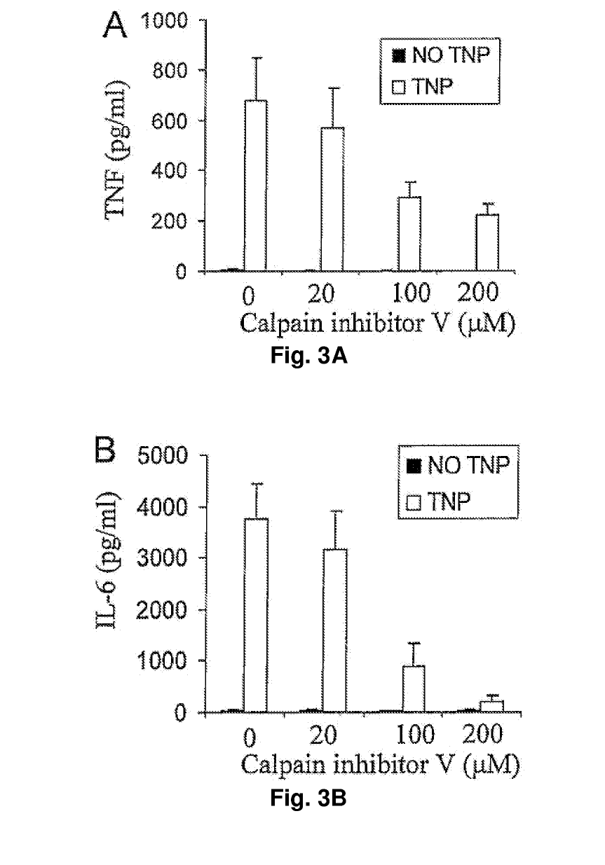

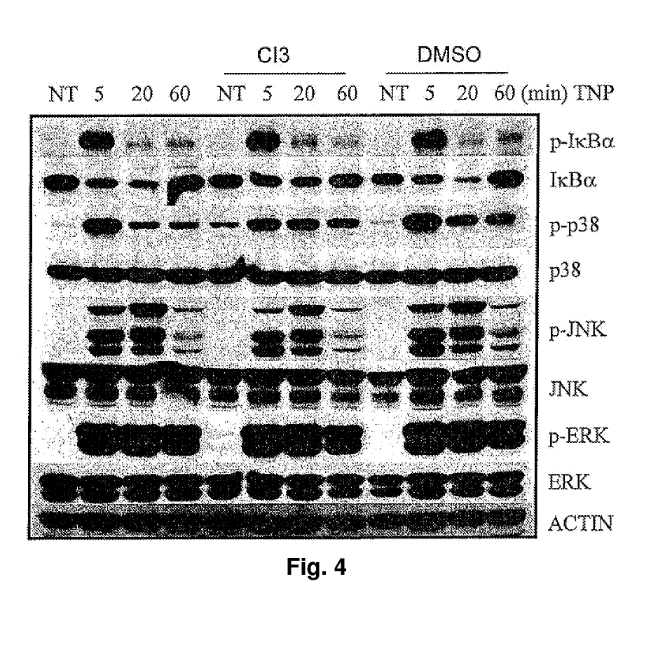

Following overnight sensitization and extensive washing, BMMC were re-suspended in RPMI 1640 supplemented with 10% FBS at a density of 0.5 million cells/ml and a total volume of 500 μl per test was aliquoted to Eppendorf tubes. An inhibitor (calpain inhibitor V) was added and samples were incubated for further 20-24 hr at 37° C. in a sterilized, humidified atmosphere containing 5% CO₂. Supernatants were then harvested and frozen for the subsequent determination of TNF (FIG. 3A) and IL-6 (FIG. 3B) concentration by ELISA according to manufacturer's protocol.

These results indicate that calpain specific inhibitors are effective at reducing inflammatory mediator release by mast cells, providing a means for reducing these effects during allergy and/or inflammation disorders.

### Example 4

### Preparation of Total Cellular Lysate

Experimental treatments leading to the acquisition of total cellular lysates for immunoblot analysis were typically carried out at densities of 1 million cells/ml. At the appropriate times, cells were harvested by centrifugation at 500×g for 5 min at 4° C. Cell pellets were immediately re-suspended in ice-cold lysis buffer (25 mM Tris-HCl, pH=7.5, 150 mM sodium chloride, 1 mM EDTA, 1 mM EGTA, 1% Triton X-100, 0.25% sodium deoxycholate, 0.1% SDS) containing freshly added 5 μg/ml leupeptin and pepstatin, 1 mM PMSF, 1 mM dithiothreitol, 100 μM sodium orthovanadate, 10 mM sodium fluoride, 10 μM phenylarsine oxide and 10 μg/ml aprotinin. Lysates were left on ice for at least 20 minutes and transferred to Eppendorf tubes for clarification at 15,000×g for 10 min at 4° C. to remove cellular debris.

In this particular experiment, mouse bone marrow-derived mast cells (60 million) were sensitized overnight with 90 ml fresh culture medium and 30 ml TIB141-conditioned medium enriched in IgE directed against trinitrophenyl (TNP). Cells were then washed twice with RPMI 1640 and resuspended in 10% FBS-RPMI 1640 at 1 million cells/ml. 5 ml of cells were aliquoted for each treatment. Calpain inhibitor 3 (CI3) or DMSO was added and to the sample and incubated at 37° C. in a 5% CO₂ incubator for 1 hour. Then, TNP-BSA (10 ng/ml) was added to stimulate mast cells for various times as indicated. After incubation, cells were harvested by centrifugation at 500×g for 5 min at 4° C. Cell pellets were immediately re-suspended in ice-cold lysis buffer (25 mM Tris-HCl, pH=7.5, 150 mM sodium chloride, 1 mM EDTA, 1 mM EGTA, 1% Triton X-100, 0.25% sodium deoxycholate, 0.1% SDS) containing freshly added 5 μg/ml leupeptin and pepstatin, 1 mM PMSF, 1 mM dithiothreitol, 100 μM sodium orthovanadate, 10 mM sodium fluoride, 10 μM phenylarsine oxide and 10 g/ml aprotinin. Lysates were left on ice for at least 20 minutes and transferred to Eppendorf tubes for clarification at 15,000×g for 10 min at 4° C. to remove cellular debris. Supernatants were then transferred to new tubes and used for Western blotting with antibodies to phospho-IκB, total IκB, phospho-p38, total p38, phospho-JNK, total JNK, phospho-ERK, total ERK and actin.

These results provide a mechanistic link between inhibition of calpain and a reduced inflammatory response and/or a decrease in mast cell mediator secretion. This provides further support to the present invention, in which calpain inhibition can be utilized for the treatment of allergy and/or inflammation disorders.

The embodiments of the present invention in which an exclusive property or privilege is claimed are defined as follows:

1. A method of therapeutically treating an allergic disease, inflammatory disease or autoimmune disease in a subject in need thereof, comprising the step of administering to said subject a pharmaceutical composition comprising an effective amount of at least one inhibitor of calpain and a pharmaceutically acceptable carrier.

2. The method according to claim 1, wherein the administration of the effective amount of at least one calpain inhibitor results in inhibition of calpain, thereby resulting in a decrease in at least one of mast cell mediator production, mast cell mediator secretion or mast cell degranulation.

3. The method according to claim 1, wherein the calpain inhibitor is a pharmaceutical compound selected from the group consisting of peptidyl epoxides, peptidyl aldehydes, peptidyl α-ketoamides, non-peptide inhibitors, and carboxamide derivatives.

4. The method according to claim 1, wherein the calpain inhibitor is a nucleic acid capable of decreasing the level of calpain expression.

5. The method according to claim 3, wherein the nucleic acid is an antisense oligonucleotide.

6. The method according to claim 3, wherein the nucleic acid is RNA.

7. The method according to claim 1, wherein the calpain inhibitor is directed to at least one of calpain 1, calpain 2 or calpain 4.

8. The method according to claim 1, wherein the allergic disease is selected from the group consisting of allergic rhinitis, atopic eczema, asthma, conjunctivitis, and anaphylaxis.

9. The method according to claim 1, wherein the autoimmune disease is selected from the group consisting of multiple sclerosis, psoriasis, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, vasculitis, Sjogren's syndrome, nodular panarteritis, autoimmune enteropathy and proliferative glomerulonephritis.

10. A drug preparation which comprises an inhibitor of calpain or a pharmaceutically acceptable salt thereof for the treatment of diseases relating to mast cell mediator secretion.

11. The drug preparation according to claim 10, wherein the inhibitor of calpain is a pharmaceutical compound selected from the group consisting of peptidyl epoxides, peptidyl aldehydes, peptidyl α-ketoamides, non-peptide inhibitors, and carboxamide derivatives.

12. The drug preparation according to claim 10, wherein the calpain inhibitor is a nucleic acid capable of decreasing the level of calpain expression.

13. The drug preparation according to claim 12, wherein the nucleic acid is an antisense oligonucleotide.

14. The drug preparation according to claim 12, wherein the nucleic acid is RNA.

15. The drug preparation according to claim 10, wherein the inhibitor of calpain is directed to at least one of calpain 1, calpain 2 or calpain 4.

16. The drug preparation according to claim 10, wherein the disease relating to mast cell mediator secretion is selected from the group consisting of allergic rhinitis, atopic eczema, asthma, conjunctivitis, anaphylaxis, multiple sclerosis, psoriasis, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, vasculitis, Sjogren's syndrome, nodular panarteritis, autoimmune enteropathy and proliferative glomerulonephritis.

17. The drug preparation according to claim 17, further comprising a compound known to alleviate allergy and/or inflammatory symptoms.

18. The drug preparation according to claim 15, wherein the compound is a histamine receptor antagonist.

19. The drug preparation according to claim 18, wherein the histamine receptor antagonist is selected from the group consisting of tricyclic dibenzoxepins, ethanolamines, ethylenediamines, alylamines, piperazines, phenothiazines, piperidines, and phthalazinones.

20. The drug preparation according to claim 17, wherein the compound is an immunomodulatory agent.

21. The drug preparation according to claim 20, wherein the immunomodulatory agent is selected from the group consisting of glucocorticoids, immunosuppressants, aminosalicylates; steroid hormones; non-steroidal anti-inflammatory drugs (NSAIDS), sympathomimetics; and analgesics.

22. The drug preparation according to claim 17, wherein the compound is selected from the group consisting of omalizumab, leukotriene receptor antagonists, leukotriene synthesis inhibitors, cromolyn, and nedocromil

23. Use of the drug preparation according to claim 10.

24. A method of decreasing degranulation of mast cells, comprising the step of decreasing the activity of calpain within mast cells.

25. The method according to claim 24, wherein the activity of the calpain is decreased through the actions of a calpain inhibitor.

26. The method according to claim 25, wherein the inhibitor is a pharmaceutical compound selected from the group consisting of peptidyl epoxides, peptidyl aldehydes, peptidyl α-ketoamides, non-peptide inhibitors, and carboxamide derivatives.

27. The method according to claim 25, wherein the inhibitor is a nucleic acid capable of decreasing the level of calpain expression.

28. The method according to claim 24, wherein the calpain is at least one of calpain 1, calpain 2 or calpain 4.

29. A method of decreasing cytokine production by mast cells, comprising the step of decreasing the activity of calpain within mast cells.

30. The method according to claim 29, wherein the activity of the calpain is decreased through the actions of a calpain inhibitor.

31. The method according to claim 30, wherein the inhibitor is a pharmaceutical compound selected from the group consisting of peptidyl epoxides, peptidyl aldehydes, peptidyl α-ketoamides, non-peptide inhibitors, and carboxamide derivatives.

32. The method according to claim 30, wherein inhibitor the inhibitor is a nucleic acid capable of decreasing the level of calpain expression.

33. The method according to claim 29, wherein the calpain is at least one of calpain 1, calpain 2 or calpain 4.
